(19) United States
(12) Patent Application Publication
(10) Pub. No.: US 2025/0090174 A1
(43) Pub. Date: Mar. 20, 2025

(54) LEFT ATRIAL APPENDAGE CLOSURE DEVICE WITH DRUG-ELUTING COMPOSITION

(71) Applicant: BOSTON SCIENTIFIC SCIMED, INC., MAPLE GROVE, MN (US)

(72) Inventors: Steven L Kangas, Woodbury, MN (US); Edward Kopesky, Mahtomedi, MN (US); Amod Shridhar Modak, Maple Grove, MN (US); Victoria Szlag, New Hope, MN (US)

(73) Assignee: BOSTON SCIENTIFIC SCIMED, INC., MAPLE GROVE, MN (US)

(21) Appl. No.: 18/888,648

(22) Filed: Sep. 18, 2024

Related U.S. Application Data
(60) Provisional application No. 63/584,094, filed on Sep. 20, 2023.

(51) Int. Cl. A61B 17/12

(52) U.S. Cl. CPC A61B 17/12031; A61B 17/12122; A61B 17/12131

(57) ABSTRACT

Medical devices as wells as methods for making and using medical devices are disclosed. An example medical device may include a left atrial appendage device. The occlusive medical device includes an expandable frame configured to shift between a first configuration and an expanded configuration and a fabric disposed along at least a portion of the expandable frame. A drug eluting coating is disposed on the fabric, the drug eluting coating including a pharmaceutically active component dispersed within a polymeric carrier.

### CROSS REFERENCE TO RELATED APPLICATIONS

This application claims the benefit of priority of U.S. Provisional Application No. 63/584,094 filed Sep. 20, 2023, the entire disclosure of which is hereby incorporated by reference.

### TECHNICAL FIELD

The present disclosure pertains to medical devices, and methods for manufacturing medical devices. More particularly, the present disclosure pertains to occlusive devices such as those deployed adjacent to the left atrial appendage.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in a an occlusive medical device. The occlusive medical device includes an expandable frame configured to shift between a first configuration and an expanded configuration and a fabric disposed along at least a portion of the expandable frame. A drug eluting coating is disposed on the fabric, the drug eluting coating including a pharmaceutically active component dispersed within a polymeric carrier.

Alternatively or additionally, the drug eluting coating may include 1 wt. % to 50 wt. % pharmaceutically active component and 99 wt. % to 50 wt. % polymeric carrier.

Alternatively or additionally, the drug eluting coating may include a drug density ranging from 0.1 μg/mm² to 20 μg/mm².

Alternatively or additionally, the drug eluting coating may include more than 10 wt. % pharmaceutically active component and less than 90 wt. % polymeric carrier.

Alternatively or additionally, the drug eluting coating may include a drug density ranging from 5 μg/mm² to 20 μg/mm².

Alternatively or additionally, the drug eluting coating may include less than 10 wt. % pharmaceutically active component and more than 90 wt. % polymeric carrier.

Alternatively or additionally, the drug eluting coating may include a drug density ranging from 0.1 μg/mm² to 2 μg/mm².

Alternatively or additionally, the pharmaceutically active component may include an anticoagulant drug.

Alternatively or additionally, the pharmaceutically active component may include rivaroxaban.

Alternatively or additionally, the polymeric carrier may include a polyvinylidene fluoride copolymer.

Alternatively or additionally, the polymeric carrier may include poly(vinylidene fluoride-co-hexafluoropropylene).

Alternatively or additionally, the occlusive medical device may further include a top coat disposed over the drug eluting coating.

Alternatively or additionally, the top coat may include a polyvinylidene fluoride copolymer.

Alternatively or additionally, wherein the drug eluting coating may be adapted to provide a release rate in a range of 5 ng/mm²/day to 250 ng/mm²/day measured at 7 days post-implantation.

Another example may be found in an occlusive medical device. The occlusive medical device includes an expandable frame configured to shift between a first configuration and an expanded configuration and a fabric disposed along at least a portion of the expandable frame. A drug eluting coating is disposed on the fabric, the drug eluting coating including rivaroxaban dispersed within a polyvinylidene fluoride copolymer. A top coat is disposed over the drug eluting coating.

Alternatively or additionally, the drug coating may include rivaroxaban dispersed within poly(vinylidene fluoride-co-hexafluoropropylene).

Alternatively or additionally, the top coat may include a polyvinylidene fluoride copolymer.

Alternatively or additionally, the top coat may include poly(vinylidene fluoride-co-hexafluoropropylene).

Alternatively or additionally, the top coat may be substantially free of a pharmaceutically active component.

Another example may be found in an occlusive medical device. The occlusive medical device includes an expandable frame configured to shift between a first configuration and an expanded configuration and a fabric disposed along at least a portion of the expandable frame. A drug eluting coating including rivaroxaban dispersed within poly(vinylidene fluoride-co-hexafluoropropylene) is disposed on the fabric. A top coat including poly(vinylidene fluoride-co-hexafluoropropylene) is disposed over the drug eluting coating.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a plan view of an illustrative medical device;

FIG. 2 is a perspective view of an illustrative medical device;

FIG. 4 is a side view of a portion of an illustrative medical device;

FIG. 5 is a side view of a portion of an illustrative medical device;

FIG. 6 is a cross-sectional view of a portion of an illustrative medical device;

FIG. 7 is a cross-sectional view of a portion of an illustrative medical device;

FIGS. 8A and 8B are schematic views of illustrative healing scenarios;

FIG. 9 is a schematic view of an illustrative healing scenario;

FIG. 10 is a schematic cross-sectional view of a portion of an illustrative occlusive member;

FIG. 11 is a schematic cross-sectional view of a portion of an illustrative occlusive member;

FIG. 12 is a graphical representation of drug release rate versus time for a high dose, slow release example;

FIG. 13 is a graphical representation of drug release rate versus time for a medium dose, slow release example;

FIG. 14 is a graphical representation of drug release rate versus time for a low dose, slow release example;

FIG. 15 is a graphical representation of drug release rate versus time for a low dose, fast release example;

FIG. 16 is a graphical representation of drug release rate versus time for a very low dose, very fast release example;

FIG. 17 is a graphical representation of drug release versus time for a low dose, low release example;

FIG. 18 is a graphical representation of clot weight versus time for several pharmaceutically active components; and

FIGS. 19A through 19D are graphical representations of percent drug released versus time for various polymeric carriers.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

The left atrial appendage (LAA) is a small sac attached to the left atrium of the heart as a pouch-like extension. In patients suffering from atrial fibrillation, the left atrial appendage may not properly contract with the left atrium, causing stagnant blood to pool within its interior, which can lead to the undesirable formation of thrombi within the left atrial appendage. Thrombi forming in the left atrial appendage may break loose from this area and enter the blood stream. Thrombi that migrate through the blood vessels may eventually plug a smaller vessel downstream and thereby contribute to stroke. Clinical studies have shown that the majority of blood clots in patients with atrial fibrillation are found in the left atrial appendage. As a treatment, medical devices have been developed which are positioned in the left atrial appendage and deployed to close off the ostium of the left atrial appendage. Over time, the exposed surface(s) spanning the ostium of the left atrial appendage becomes covered with tissue (a process called endothelization), effectively removing the left atrial appendage from the circulatory system and reducing or eliminating the number of thrombi which may enter the blood stream from the left atrial appendage. In an effort to reduce the occurrence of thrombi formation within the left atrial appendage and prevent thrombi from entering the blood stream from within the left atrial appendage, it may be desirable to develop medical devices and/or occlusive implants that close off the left atrial appendage from the heart and/or circulatory system, thereby lowering the risk of stroke due to thrombolytic material entering the blood stream from the left atrial appendage. Example medical devices and/or occlusive implants which seal the left atrial appendage (or other similar openings) are disclosed herein.

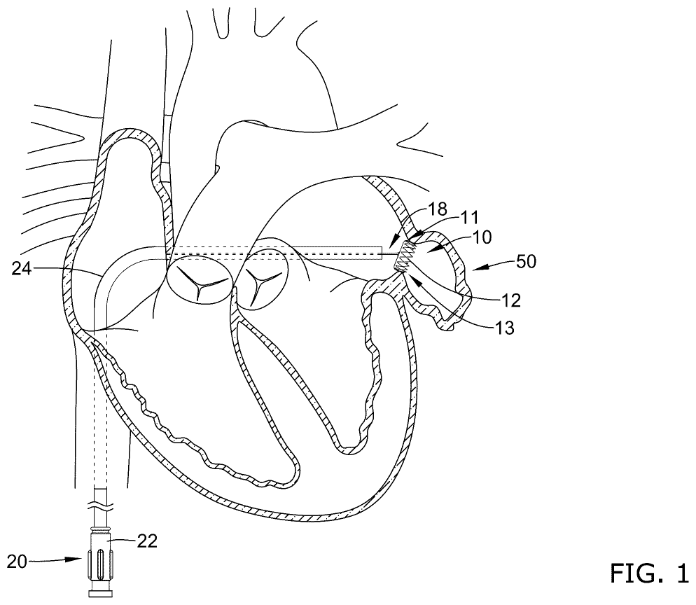

FIG. 1 illustrates an example occlusive implant 10 (e.g., a left atrial appendage medical device) positioned within the left atrial appendage 50. FIG. 1 further illustrates that the occlusive implant 10 may be inserted and advanced through a body lumen via an occlusive implant delivery system 20. In some instances, an occlusive implant delivery system 20 may include a delivery catheter 24 which is guided toward the left atrium via various chambers and lumens of the heart (e.g., the inferior vena cava, superior vena cava, the right atrium, etc.) to a position adjacent the left atrial appendage 50.

The delivery system 20 may include a hub 22. The hub 22 may be manipulated by a clinician to direct the distal end region of the delivery catheter 24 to a position adjacent the left atrial appendage 50. In some instances, an occlusive implant delivery system 20 may include a core wire 18. Further, a proximal end region 11 of the occlusive implant 10 may be configured to releasably attach, join, couple, engage, or otherwise connect to the distal end of the core wire 18. In some instances, the proximal end region 11 of the occlusive implant 10 may include a threaded insert coupled thereto. In some instances, the threaded insert may be configured to and/or adapted to couple with, join to, mate with, or otherwise engage a threaded member disposed at the distal end of a core wire 18. Other structures for releasably coupling and/or engaging the proximal end of the occlusive implant 10 to the distal end of the core wire 18 are also contemplated.

FIG. 1 further illustrates the occlusive implant 10 positioned adjacent the left atrial appendage 50 via the delivery catheter 24 (described above). It can be appreciated that in some examples, the occlusive implant 10 may be configured to shift between a first or collapsed configuration and a second or expanded configuration. For example, in some instances, the occlusive implant 10 may be in a collapsed configuration during delivery via the occlusive implant delivery system 20, whereby the occlusive implant 10 expands to an expanded configuration once deployed from the occlusion implant delivery system 20.

Additionally, FIG. 1 illustrates that the occlusive implant 10 may include an expandable frame or framework 12. The expandable framework 12 may be compliant and, therefore, substantially conform to and/or be in sealing engagement with the shape and/or geometry of a lateral wall of a left atrial appendage 50 in the expanded configuration. In some embodiments, the occlusive implant 10 may expand to a size, extent, or shape less than or different from a maximum unconstrained extent, as determined by the surrounding tissue and/or lateral wall of the left atrial appendage 50. Further, it can be appreciated that the elements of the expandable framework 12 may be tailored to increase the flexibility of the expandable framework 12 and/or the occlusive implant 10, thereby permitting the expandable framework 12 and/or the occlusive implant 10 to conform to the tissue around it, rather than forcing the tissue to conform to the expandable framework 12 and/or the occlusive implant 10. Additionally, in some instances, it may be desirable to design the occlusive implant 10 to include various features, components and/or configurations which improve the sealing capabilities of the occlusive implant 10 within the left atrial appendage.

FIG. 1 illustrates that the distal end region 13 of the expandable framework 12 may extend farther into the left atrial appendage 50 as compared to the proximal end region 11 of the expandable framework 12. It can be appreciated that as the expandable framework 12 is advanced into the left atrial appendage 50, the distal end region 13 may engage with tissue defining the left atrial appendage 50. In other words, in some examples the distal end region 13 may be considered the “leading” region of the expandable framework 12 as it enters into the left atrial appendage 50. However, this is not intended to be limiting. Rather, in some examples the proximal end region 11 may be considered the “leading” region of the expandable framework 12 as it enters into the left atrial appendage 50.

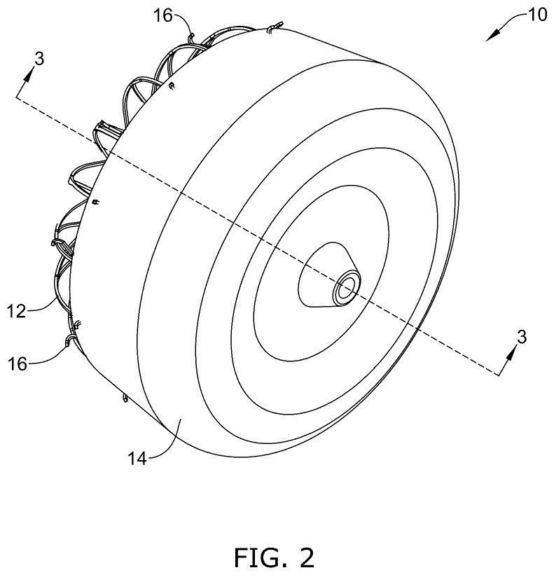

FIG. 2 is a perspective view of an example occlusive implant 10 and FIG. 3 is a cross-sectional view taken along the line 3-3 of FIG. 2. The occlusive implant 10 may include an expandable framework 12. The expandable framework 12 may include a proximal end region 11 and a distal end region 13. FIGS. 2 and 3 illustrate that the proximal end region 11 of the expandable framework 12 may include a plurality of support members 19 extending circumferentially around the longitudinal axis 52 of the expandable framework 12. FIG. 3 illustrates that that plurality of support members 19 may include one or more curved portions which are shaped such that they define a “recess” 21 extending distally into the expandable framework 12. As illustrated in FIG. 3, the recess 21 may extend circumferentially around the longitudinal axis 52. Further, FIG. 3 illustrates that each of the plurality of support members 19 may include a first end 25 which is attached to a central hub 23. It can be appreciated that the central hub 23 may be aligned along the longitudinal axis 52 of the expandable framework 12. As will be described in greater detail below, FIG. 3 illustrates that the hub 23 may be positioned such that it lies within the recess 21 defined by the plurality of support members 19.

The occlusive implant 10 may also include an occlusive member 14 disposed on, disposed over, disposed about, or covering at least a portion of the expandable framework 12. In some instances, the occlusive member 14 may be disposed on, disposed over, disposed about or cover at least a portion of an outer (or outwardly-facing) surface of the expandable framework 12. FIG. 2 further illustrates that the occlusive member 14 may extend only partially along the longitudinal extent of the expandable framework 12. However, this is not intended to be limiting. Rather, the occlusive member 14 may extend along the longitudinal extent of the expandable framework 12 to any degree (e.g., the full longitudinal extend of the expandable framework 12).

In some embodiments, the occlusive member 14 may be permeable or impermeable to blood and/or other fluids, such as water. In some embodiments, the occlusive member 14 may include a woven fabric/material or mesh, a non-woven fabric/material or mesh, a braided and/or knitted material, a fiber, a sheet-like material, a fabric, a mesh, a fabric mesh, a polymeric membrane, a metallic or polymeric mesh, a porous filter-like material, a covering, and/or other suitable construction. In some embodiments, the occlusive member 14 may prevent thrombi (i.e. blood clots, etc.) from passing through the occlusive member 14 and out of the left atrial appendage into the blood stream. In some embodiments, the occlusive member 14 may promote endothelialization after implantation, thereby effectively removing the left atrial appendage from the patient's circulatory system. Some suitable, but non-limiting, examples of materials for the occlusive member 14 are discussed below.

FIG. 2 further illustrates that the expandable framework 12 may include a plurality of anchor members 16 disposed about a periphery of the expandable framework 12. The plurality of anchor members 16 may extend radially outward from the expandable framework 12. In some embodiments, at least some of the plurality of anchor members 16 may each have and/or include a body portion and a tip portion projecting circumferentially therefrom, as shown in FIG. 2. Some suitable, but non-limiting, examples of materials for the expandable framework 12 and/or the plurality of anchor members 16 are discussed below.

In some examples, the expandable framework 12 and the plurality of anchor members 16 may be integrally formed and/or cut from a unitary member. In some embodiments, the expandable framework 12 and the plurality of anchor members 16 may be integrally formed and/or cut from a unitary tubular member and subsequently formed and/or heat set to a desired shape in the expanded configuration. In some embodiments, the expandable framework 12 and the plurality of anchor members 16 may be integrally formed and/or cut from a unitary flat member, and then rolled or formed into a tubular structure and subsequently formed and/or heat set to the desired shape in the expanded configuration. Some exemplary means and/or methods of making and/or forming the expandable framework 12 include laser cutting, machining, punching, stamping, electro discharge machining (EDM), chemical dissolution, etc. Other means and/or methods are also contemplated.

As illustrated in FIG. 3, the plurality of anchor members 16 disposed along the expandable framework 12 may include two rows of anchor members 16. However, this is not intended to be limiting. Rather, the expandable framework 12 may include a single row of anchor members 16. In other examples, the expandable framework 12 may include more than two rows of anchor members 16. For example, in some instances the expandable framework 12 may include 1, 2, 3, 4 or more rows of anchor members 16.

While FIG. 3 illustrates an expandable framework 12 which may be formed from a unitary member, this is not intended to be limiting. Rather, it is contemplated the expandable framework 12 may include a variety of different configurations which may be formed via a variety of manufacturing techniques.

As indicated above, the occlusive member 14 may include a woven fabric/material or mesh, a non-woven fabric/material or mesh, a braided and/or knitted material, a fiber, a sheet-like material, a fabric, a mesh, a fabric mesh, a polymeric membrane, a metallic or polymeric mesh, a porous filter-like material, a covering, and/or other suitable construction. The occlusive member 14 may be formed from a suitable material such as polyethylene terephthalate, polyester, nylon, acrylic materials, a polyolefin, and/or the like, combinations thereof, and/or other materials disclosed herein. In other instances, the occlusive material may include metallic mesh formed from nickel-titanium alloy, stainless steel, titanium, other materials disclosed herein, combinations thereof, and/or the like.

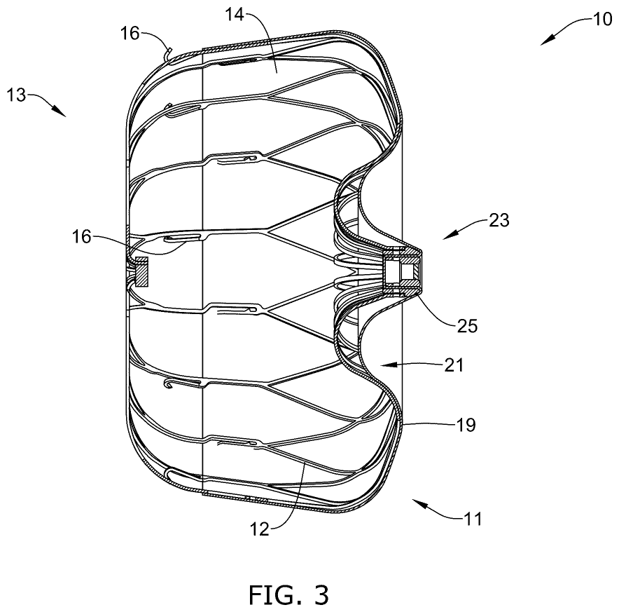

A portion of the occlusive member 14 is shown in FIG. 4. Here it can be seen that the occlusive member 14 may include one or more bundles of filaments or fibers, which may also be termed fiber bundles 54. The fiber bundles 54 may have a size/diameter in the range of about 10-500 μm, or about 20-200 μm, or about 50-150 μm, or about 100 μm. These are just examples. Other numbers are contemplated. Each of the fiber bundles 54 may include a suitable number of individual filaments 56. For example, each of the fiber bundles 54 may include 2-100 filaments 56, or about 5-50 filaments, or about 5-30 filaments, or about 10-25 filaments, or about 15-20 filaments. These are just examples. Other numbers are contemplated. In some instances, the individual filaments 56 may have a size/diameter in the range of about 1-100 μm, or about 2-25 μm, or about 2-20 μm, or about 5-15 μm, or about 10 μm. These are just examples. Other numbers are contemplated. The one or more fiber bundles 54 may be arranged to form the fabric mesh structure of the occlusive member 14. This may include braiding, knitting, weaving, e-spinning, or otherwise arranging the fiber bundles 54 into the desired arrangement or pattern.

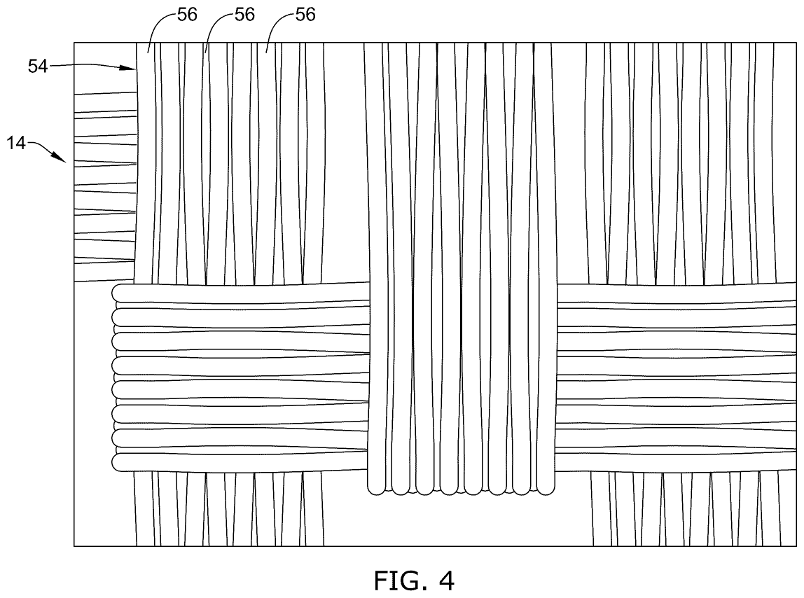

In some instances, the filaments 56 may be surface treated. For example, the filaments can be plasma treated, laser etched, and/or the like. This may improve adhesion of a drug eluting coating 58 (FIG. 5) and/or increase the surface hydrophobicity (which may improve anti-thrombogenicity), for example by imparting a nanostructure onto the filaments 56, which when coated with a drug eluting coating 58 can lead to a superhydrophobic surface.

As indicated above, the occlusive member 14 may prevent thrombi (i.e. blood clots, etc.) from passing through the occlusive member 14 and out of the left atrial appendage into the blood stream. In some instances, thrombus can form along, for example, the atrial face of the occlusive member 14.

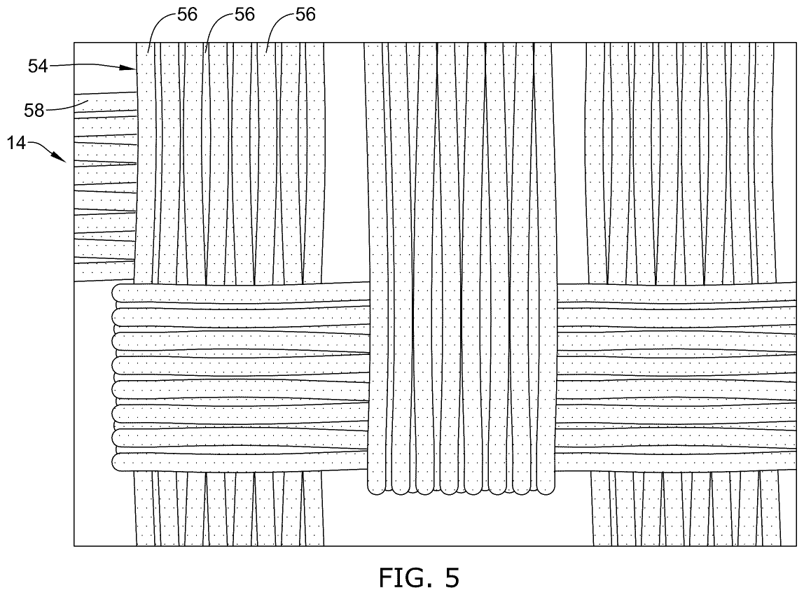

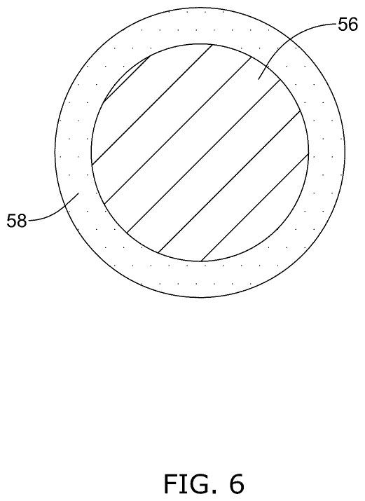

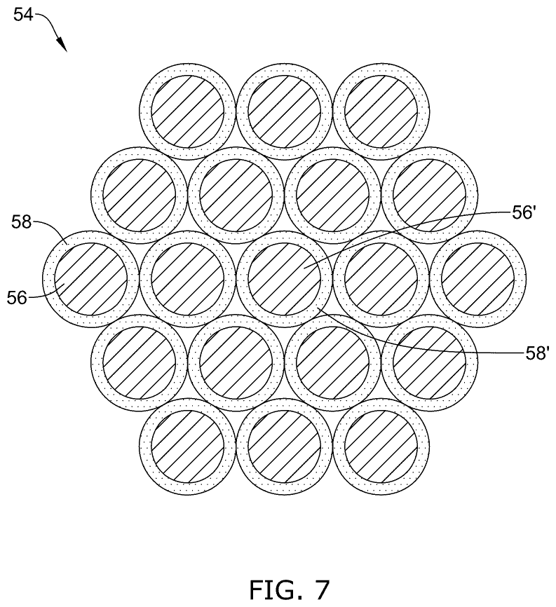

Because of the structure of the occlusive member 14, the drug eluting coating 58 may efficiently coat throughout the fabric mesh. For example, the drug eluting coating 58 may wick up throughout the occlusive member 14 (e.g., due to capillary action). When doing so, the drug eluting coating may cover, surround, and/or otherwise encapsulate each of the filaments 56 of the fiber bundles 54 as depicted in FIG. 6. The drug eluting coating 58 may be substantially uniform along the occlusive member 14. When the drug eluting coating 58 is applied via a dip coating process, it would be expected that gravitational forces result in a non-uniform coating thickness. Surprisingly, however, the structure of the occlusive member 14 results in the drug eluting coating 58 wicking along the filaments 56 and being held in place. Thus, substantially uniform coating thicknesses can be achieved, even when the anti-thrombogenic coating 58 is applied via dip coating. Furthermore, the drug eluting coating 58 may penetrate between and through the fiber bundles 54. In doing so, the drug eluting coating 58 may reach an interior region of the fiber bundles 54. For example, FIG. 7 depicts a fiber bundle 54 with an interior filament 56′ including the drug eluting coating 58′.

In some instances, the drug eluting coating 58 is disposed along only the occlusive member 14. In such instances, the expandable framework 12 may be substantially free of the drug eluting coating 58. For example, the drug eluting coating 58 may be applied to the occlusive member 14 prior to the occlusive member 14 being secured to the expandable framework 12. This, however, is not intended to be limiting. In some instances, the drug eluting coating 58 may be disposed along portions or all of the expandable framework 12. Thus, the expandable framework 12 may include the drug eluting coating 58. In some instances, the expandable framework 12 may be surface treated (e.g., plasma treated, laser etched, and/or the like). This may improve adhesion of the drug eluting 58 and/or increase the surface hydrophobicity. In some instances, a polymeric top coat (not shown) may be applied over the drug eluting coating 58.

Because of the desirable coating/adhesion of the drug eluting coating 58 to the filaments 56, the drug eluting coating 58 may demonstrate good integrity. As an example, should any portions of the drug eluting coating 58 fail (e.g., become disassociated and/or delaminated from the fiber bundles 54/filaments 56), for example due to loading and/or to repeated deployments), the failure may be limited to along an individual filament 56 rather than the drug eluting coating 58 as a whole. Because of this, the majority of the occlusive member 14 can maintain its anti-thrombogenic properties even if part of the drug eluting coating 58 breaches from the occlusive member 14.

The structure of the occlusive member 14 utilizing fiber bundles 54 formed from a plurality of individual filaments 56 may enhance wicking capillary action (e.g., relative to a “monofilament” design) of the drug eluting coating 58. For example, the increased surface area of the fiber bundles (e.g., due to the filaments 56) may allow the drug eluting coating 58 to coat, penetrate, and surround/encapsulate the filaments 56. The resulting drug eluting coating 58 may have a thickness on the order of about 20-200 nm or so while stile covering/surrounding/encapsulating essentially each of the filaments 56 in substantially their entirety. In some instances, the drug eluting coating 58 itself may have little or no impact on the mechanical properties of the occlusive member 14.

In some instances, there may be a desire to provide the occlusive implant 10 with a drug eluting composition 58 that elutes a pharmaceutically active component at a rate that initially prevents large runaway thrombus but then slows to a release rate that allows for a provisional thrombus to form, as this can be beneficial in providing a base matrix for subsequent healing. Duration of release is also an important parameter. There may be a desire to have a release profile that is long enough to get past an inflammatory state induced by implantation of the occlusive implant 10, but not so long as to unnecessarily prolong healing.

In some instances, too high of a drug release rate results in little to no healing. In some instances, too low of a drug release rate results in too much thrombus formation. In some instances, there may be a trade-off between wanting to encourage tissue growth while limiting excessive thrombus formation. As an example, a drug eluting coating may be adapted to provide a release rate in a range of 5 ng/mm²/day to 200 ng/mm²/day measured at 7 days post-implantation.

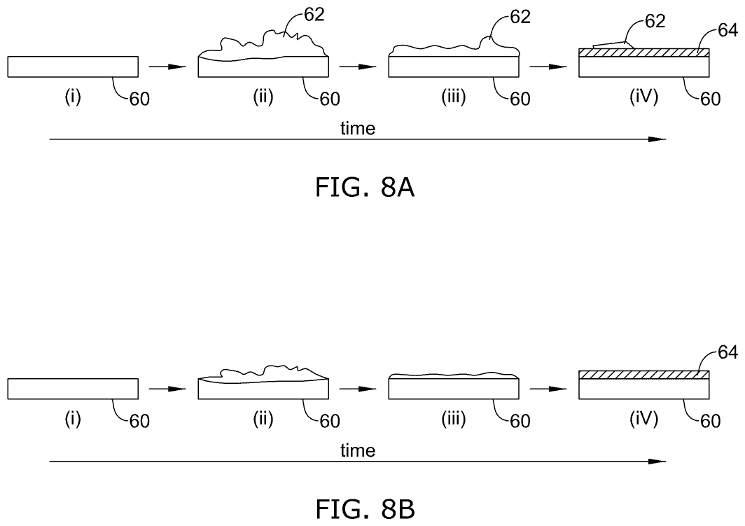

FIGS. 8A and 8B are schematic views showing differences in healing scenarios. FIG. 8A shows a typical response observed in a canine model (no systemic anticoagulation) to the occlusive implant 10, shown as having a PET (polyethylene terephthalate) fabric layer 60. Fourteen (14) days after implantation, TEE imaging can show varying levels of significant thrombus on the face (fabric) of the occlusive implant 10, as schematically shown as a thrombus 62 at time (ii). By 28 days, the thrombus 62 has reduced in size, as schematically shown at (iii). By 45 days the devices are mostly healed, with some devices showing some level of residual thrombus 62 and a healed tissue layer 64, as schematically shown at (iv). The fibrin thrombus converts to the collagenous-like tissue layer 64. FIG. 7B shows a response in which there is a smaller initial thrombus, as schematically shown at (ii). By 45 days, as schematically shown at (iv), the healed tissue layer 64 does not have any remaining thrombus 62 (in comparison to FIG. 8A).

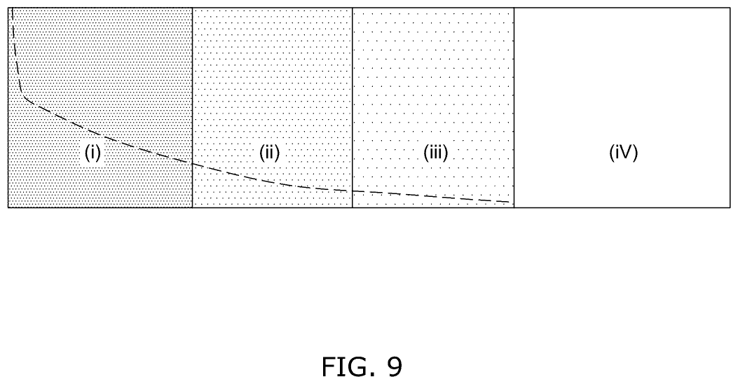

FIG. 9 is a schematic view showing the effect of having an occlusive implant 10 that is adapted to elute a pharmaceutically active component such as rivaroxaban. The initial release phase (I) is characterized by high release (surface burst) and high local concentration of drug at the surface of the occlusive implant 10 resulting in essentially no thrombus formation. In this phase, the face of the occlusive implant 10 is essentially “immune” from thrombus formation (and healing). In phase II, the release rate has slowed to a level that some low level of provisional thrombus can form, leading to the initiation of the healing process, although the local surface concentration of drug is still high enough to prevent runaway thrombus formation. In phase III, the drug release slowly goes to zero. By this time, healing is well underway and the risk of thrombus formation on the face of the occlusive implant 10 is very low. The duration of each one of these release phases impacts risk of thrombus and speed of healing. For example, if the duration of phase I is too long, then subsequent healing will be delayed. If phase I-IV are too short then there is higher risk of runaway thrombus formation after the drug is done eluting because local trauma due to the implant procedure has not resolved, leaving the patient in a hypercoagulable state.

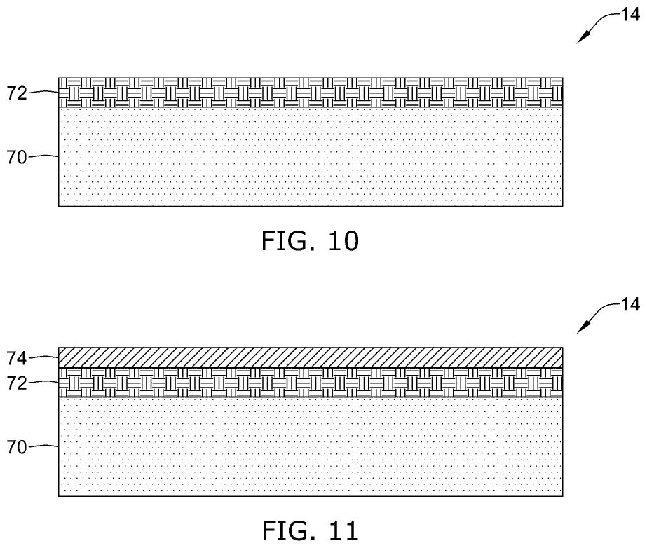

FIG. 10 is a schematic view of the occlusive member 14 that may form a part of the occlusive implant 10. The occlusive member 14 includes a fabric 70. In some instances, as shown in previous Figures, the fabric 70 may include fiber bundles such as the fiber bundles 54, that each include a number of individual filaments such as the filaments 56. A drug eluting coating 72 is disposed on the fabric 70. The drug eluting coating 72 may be representative of the drug eluting coating 58, for example. The drug eluting coating 72 may surround each fiber bundle 54 or even each filament 56. In some instances, the drug eluting coating 72 may form a solid layer over the fabric 70. The drug eluting coating 72 may include a pharmaceutically active component dispersed within a polymeric carrier.

In FIG. 11, a polymeric top coat 74 has been disposed over the drug eluting coating 72. In some instances, inclusion of the polymeric top coat 74 may slow down the release of the pharmaceutically active component or components within the drug eluting coating 72. In some instances, the polymeric top coat 74 may surround the drug eluting coating 72 as the drug eluting coating 72 surrounds each fiber bundle 54 or filament 56. The polymeric top coat 74 may form a solid layer over the drug eluting coating 72, and also the fabric 70.

In some instances, the polymeric top coat 74 may be free of, or substantially free, of any pharmaceutically active components. Substantially free may be interpreted as referencing no intentionally added pharmaceutically active components within the polymeric top coat 74, with the understanding that during formation of the drug eluting coating 72 and the polymeric top coat 74 that there may be unintentional diffusion of molecules of the pharmaceutically active components from within the drug eluting coating 72 and into the polymeric top coat 74. FIGS. 10 and 11 may be considered as being on a macro scale, with the fabric 70 spanning the face of the occlusive implant 10. FIGS. 10 and 11 may be considered as being on a micro scale, with the fabric 70 representing a single fiber bundle 54, or even a single filament 56.

In some instances, the drug eluting coating 72 may have a thickness sufficient to provide a desired drug density value, or to provide sufficient pharmaceutically active component to elute over a desired period of time. In some instances, the drug eluting coating 72 may have an average thickness in a range of 0.1 μm to 25 μm, for example. In some instances, the polymeric top coat 74 may have a thickness sufficient to slow a drug release rate to a desired rate. In some instances, the polymeric top coat 74 may have an average thickness in a range of 0.1 μm to 15 μm, for example.

The drug eluting coating 72 may include one or more pharmaceutically active components that are dispersed within a polymeric carrier. The drug eluting coating 72 may be applied to the occlusive member 14 using a suitable method such as dip coating, spray coating, or the like. This may include dissolving the materials used to form the drug eluting coating 58, such as one or more pharmaceutically active components and a polymeric carrier, into a suitable solvent or solvent mix, in order to form a dilute solution (e.g., 0.05%-2% solids) and applying (e.g., by dipping, spraying, etc.) the drug eluting coating 58 to the occlusive member 14.

Examples of suitable pharmaceutically active components include DOAC (direct oral anticoagulants) components. Examples of DOAC components include the factor Xa inhibitors Apixaban, which is sold under the brand name Eliquis; Rivaroxaban, which is sold under the brand name Xarelto; and Edoxaban, which is sold under the brand name Savaysa. The chemical name for rivaroxaban is 5-Chloro-N-({(5S)-2-oxo-3-[4-(3-oxo-4-morpholinyl)phenyl]-1,3-oxazolidin-5-yl}methyl)-2-thiophenecarboxamide. Rivaroxaban has the following chemical structure:

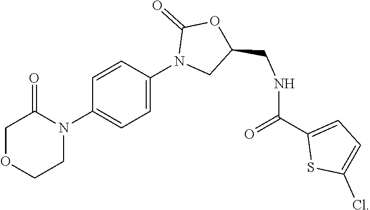 [SMILES: O=C(NC[C@H]1CN(c2ccc(N3CCOCC3=O)cc2)C(=O)O1)c1ccc(Cl)s1]

Examples of other factor Xa inhibitors that may be used include Betrixaban, Antistasin, TAP (tick anticoagulant peptide), DX-9065a, YM-60828, Lataxaban, and eribaxaban.

The polymeric carrier may include a single polymer, or a mix of two or more polymers. The polymeric carrier may include a block copolymer or random copolymer, for example. The polymeric carrier may include any of a variety of different polymers, including but not limited to a fluoropolymer, polyvinylidene fluoride, a polyvinylidene fluoride (PVDF) copolymer, poly(vinylidene fluoride-co-hexafluoropropylene) (PVDF-HFP), fluorine functional phosphazene polymers, poly[bis(trifluoroethoxy)phosphazene], polytetrafluoroethylene, polytetrafluoroethylene copolymers, combinations thereof, and/or the like. Other polymers are also contemplated, including styrene-isobutylene-styrene block (SIBS) block terpolymer and polybutylmethacrylate (PBMA). Additional polymers that may be used as the polymeric carrier include ethylene vinyl acetate copolymers, polybutylmethacrylate, acrylic copolymers, PVP copolymers, polyvinyl acetate, polyurethanes, phosphorylcholine polymers and biodegradable polymers such as PLGA (poly(lactic-co-glycolic acid), PLA (polylactic acid), polycaprolactone polymers and copolymers thereof.

The polymeric top coat 74 may be formed from any of a variety of different polymers. In some instances, the polymeric top coat 74 may be formed from the same polymer or polymers used as the polymeric carrier for the drug eluting coating 72. As an example, the polymeric top coat 74 may include a polyvinylidene fluoride copolymer. As another example, the polymeric top coat 74 may include poly(vinylidene fluoride-co-hexafluoropropylene). In some instances, the polymeric top coat 74 may include any of a variety of different polymers, including but not limited to a fluoropolymer, polyvinylidene fluoride, a polyvinylidene fluoride (PVDF) copolymer, poly(vinylidene fluoride-co-hexafluoropropylene) (PVDF-HFP), fluorine functional phosphazene polymers, poly[bis (trifluoroethoxy)phosphazene], polytetrafluoroethylene, polytetrafluoroethylene copolymers, combinations thereof, and/or the like. Other polymers are also contemplated, including styrene-isobutylene-styrene block (SIBS) block terpolymer and polybutylmethacrylate (PBMA). Additional polymers that may be used as the polymeric carrier include ethylene vinyl acetate copolymers, polybutylmethacrylate, acrylic copolymers, PVP copolymers, polyvinyl acetate, polyurethanes, phosphorylcholine polymers and biodegradable polymers such as PLGA (poly(lactic-co-glycolic acid), PLA (polylactic acid), polycaprolactone polymers and copolymers thereof.

Any of a variety of solvents may be used. Illustrative solvents include but are not limited to acetone and DMF (dimethylformamide). Other example solvents are DMSO (dimethylsulfoxide), THF (tetrafhydrofuran), cyclohexanone, DMAc (dimethylacetamide), ethyl acetate, NMP (n-methylpyrrolidone), MEK (methyl ethyl ketone), MIBK (methyl isobutyl ketone) and ACN (acrylonitrile). In some instances, a single solvent may be used for dissolving both the polymeric carrier and the pharmaceutically active component or components. In some instances, a first solvent may be used to dissolve the pharmaceutically active component or components, and a second solvent may be used to dissolve the polymeric carrier. The first solvent including the dissolved pharmaceutically active component or components and the second solvent including the dissolved polymeric carrier may be mixed together in order to form a coating composition for forming the drug eluting layer 72. The coating composition may then be used to form the drug eluting coating 72, such as via dip coating or spray coating. One or more solvents may be used in dissolving the polymer or polymers used to form the polymeric top coat 74 to form a top coat composition. The top coat composition may then be used to form the polymeric top coat 74 over the drug eluting coating 72, such as being dip coating or spray coating.

In some instances, the drug eluting coating 72 may include 1 wt. % to 50 wt. % pharmaceutically active component and 99 wt. % to 50 wt. % polymeric carrier. In some instances, the drug eluting coating 72 may have a drug density ranging from 0.1 μg/mm² to 20 μg/mm². As an example, the drug eluting coating 72 may include more than 10 wt. % pharmaceutically active component and less than 90 wt. % polymeric carrier. The drug eluting coating 72 may include a drug density ranging from 5 μg/mm² to 20 μg/mm². As another example, the drug eluting coating 72 may include less than 10 wt. % pharmaceutically active component and more than 90 wt. % polymeric carrier. The drug eluting coating 72 may have a drug density ranging from 0.1 μg/mm² to 2 μg/mm², for example.

The materials that can be used for the occlusive implant 10 may include those commonly associated with medical devices. For example, the occlusive implant 10 and/or other components thereof may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the occlusive implant 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the occlusive implant 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the occlusive implant 10 to achieve the same result.

In some embodiments, a degree of Magnetic Resonance Imaging (MRI) compatibility is imparted into the occlusive implant 10. For example, the occlusive implant 10, or portions thereof, may be made of a material that does not substantially distort the image and create substantial artifacts (e.g., gaps in the image). Certain ferromagnetic materials, for example, may not be suitable because they may create artifacts in an MRI image. The occlusive implant 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

### EXAMPLES

The disclosure may be further clarified by reference to the following Examples, which serve to exemplify some embodiments, and not to limit the disclosure in any way. The Examples explore the impact of various compositions and material thicknesses in drug release rates and corresponding impact on thrombus formation.

### Example 1—High Dose, Slow Release Occlusive Implant

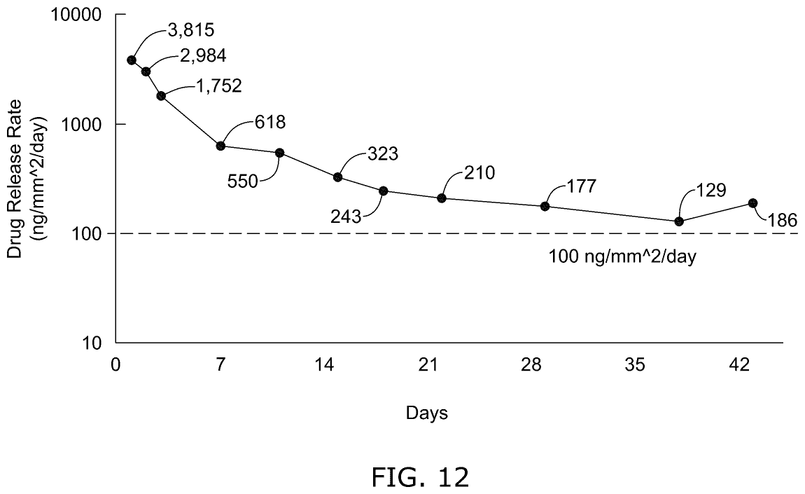

An occlusive implant (e.g., including an expandable framework and an occlusive member) was spray coated with a solution of rivaroxaban and PVDF-HFP (30:70 rivaroxaban:PVDF ratio, solvents: 60:40 acetone:DMF) to achieve an average dose density of about 15 μg/mm² on the outer face of the occlusive implant. The occlusive implants were then spray coated with a topcoat solution of PVDF-HFP in acetone. The purpose of the topcoat was to slow the rate of drug release. FIG. 12 shows that there was a significant burst of release drug over the first week, followed by a slow decay over the next month. The drug release rate never dropped below 100 ng/mm²/day within the first 45 days.

The spray coated occlusive implants were implanted for 45 days in the left atrial appendages of three different canines. The canines were given no antiplatelet and no anticoagulant medication post-implant. Transesophageal echocardioagraph (TEE) images from each dog 14 days post-implant displayed no evidence of thrombus on any of the devices, indicating that the rivaroxaban is significantly impacting the local formation of provisional thrombus on the devices. Rivaroxaban blood content analysis showed that the systemic concentration of rivaroxaban in all the canines was undetectable beyond 24 hours, indicating that the thrombus inhibition was strictly due to local action of the drug, not due to systemic effects of the drug in the bloodstream. Drug content analysis from the explant from Canine 3 indicated that approximately 15% of the initial drug remained on the device at explant, in good agreement with the in vitro drug release data that predicted 14% of the initial drug would remain at 45 days.

Computed tomography (CT) images were obtained at 45 days post-implant, showing significant flow of contrast through the mesh of the Watchman device, indicating minimal tissue coverage on the occlusive implants. The explanted occlusive implants exhibit almost no tissue coverage, indicating that a sustained release of rivaroxaban at ≥100 ng/mm²/day can inhibit provisional thrombus formation for at least 45 days in highly thrombogenic canines.

### Example 2—Medium Dose, Slow Release Occlusive Implant

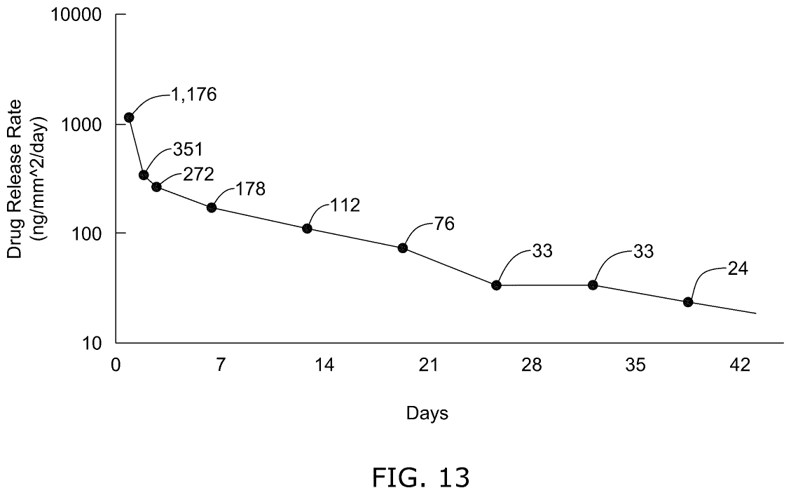

An occlusive implant (e.g., including an expandable framework and an occlusive member) was spray coated with a solution of rivaroxaban and PVDF-HFP (20:80 rivaroxaban:PVDF ratio, solvents: 47:53 acetone:DMF) to achieve an average dose density of about 5 μg/mm² on the outer face of the occlusive implant. The occlusive implants were then spray coated with a topcoat solution of PVDF-HFP in acetone. The purpose of the topcoat was to slow the rate of drug release. FIG. 13 shows that there was a significant burst of release drug over the first week, followed by a slow decay over the next month. The drug release rate never dropped below 30 ng/mm²/day within the first 35 days.

The spray coated occlusive implants were implanted for 45 days in the left atrial appendages of three different canines. The canines were given no antiplatelet and no anticoagulant medication post-implant. TEE images from each dog 14 days post-implant displayed no evidence of thrombus on any of the occlusive implants, indicating that the rivaroxaban is significantly impacting the local formation of provisional thrombus on the devices. Rivaroxaban blood content analysis showed that the systemic concentration of rivaroxaban in all the canines was undetectable beyond 24 hours, indicating that the thrombus inhibition was strictly due to local action of the drug, not due to systemic effects of the drug in the bloodstream. Drug content analysis from the explant from Canine 3 indicated that approximately 18% of the initial drug was on the device at explant, in good agreement with the in vitro drug release data that predicted 13% of the drug would be present at 90 days.

CT images were obtained at 45 days post-implant, showing significant flow of contrast through the mesh of the occlusive implants, although less contrast inflow than what was observed in the high dose occlusive implants in Example 1. The canines were survived to 90 days to provide additional time for devices to cover with tissue prior to sacrifice. The 90 day CT results look remarkably similar to the 45 day CT results, indicating minimal progression in tissue coverage between 45 days and 90 days. Examination of explanted occlusive implants after 90 days indicated large variation in tissue coverage. Canine 1 exhibited nearly 100% tissue coverage while Canine 3 exhibited only about 10% mature tissue coverage along with small amounts of red granulation tissue indicative of early stage thrombus formation. The results indicate that a sustained drug release ≥30 ng/mm²/day for at least 45 days delays the formation of complete tissue coverage, out to 90 days.

### Example 3—Low Dose, Slow Release Occlusive Implant

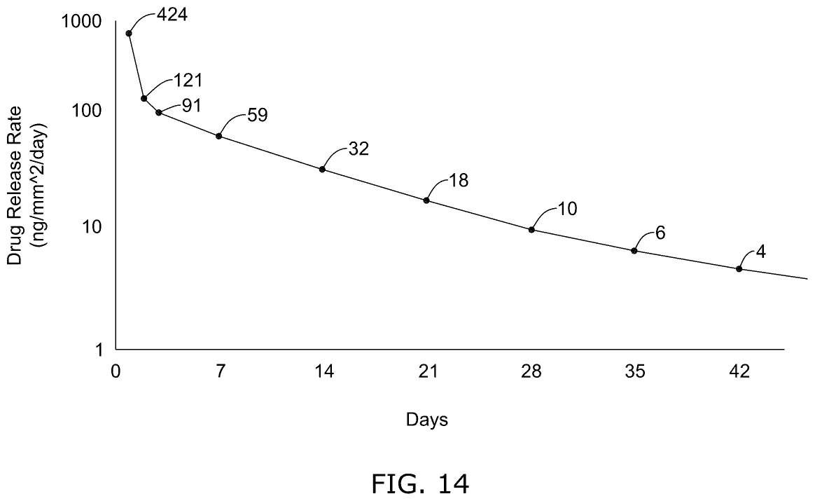

An occlusive implant (e.g., including an expandable framework and an occlusive member) was spray coated with a solution of rivaroxaban and PVDF-HFP (10:90 rivaroxaban:PVDF ratio, solvents: 30:70 acetone:DMF) to achieve an average dose density of about 1.5 μg/mm² on the outer face of the occlusive implant. The occlusive implants were then spray coated with a topcoat solution of PVDF-HFP in acetone. The purpose of the topcoat was to slow the rate of drug release. FIG. 14 shows that there was a significant burst of release drug over the first week, followed by a slow decay over the next month. The drug release rate never dropped below 30 ng/mm²/day within the first 14 days.

The spray coated occlusive implants were implanted for 90 days in the left atrial appendages of three different canines. The canines were given no antiplatelet and no anticoagulant medication post-implant. TEE images from each dog 14 days post-implant displayed no evidence of thrombus on any of the occlusive implants, indicating that the rivaroxaban is significantly impacting the local formation of provisional thrombus on the devices. Rivaroxaban blood content analysis showed that the systemic concentration of rivaroxaban in all the canines was undetectable beyond 24 hours, indicating that the thrombus inhibition was strictly due to local action of the drug, not due to systemic effects of the drug in the bloodstream. Drug content analysis from the explant from Canine 3 indicated that approximately 12% of the initial drug was on the device at explant, in good agreement with the in vitro drug release data that predicted 11% of the drug would be present at 90 days.

CT images were obtained at 45 days post-implant, showing complete occlusion in Canine 1 (minimal contrast inflow) and incomplete occlusion in Canine 2 and Canine 3 (contrast inflow through the occlusive implant mesh). Less contrast inflow relative to what was seen in Examples 1 and 2. The canines were survived to 90 days to provide additional time for occlusive implants to cover with tissue prior to sacrifice. The 90 day CT results look remarkably similar to the 45 day CT results, indicating minimal progression in tissue coverage between 45 days and 90 days. Examination of explanted occlusive implants after 90 days indicated large variation in tissue coverage. Canine 1 exhibited nearly 100% tissue coverage, Canine 2 exhibited about 90% tissue coverage and Canine 3 exhibited about 50% tissue coverage. The results indicate that a sustained drug release ≥30 ng/mm²/day for at least 14 days, along with a slow decay in drug release beyond 14 days, yields almost complete tissue coverage at 90 days.

### Example 4—Low Dose, Fast Release Occlusive Implant

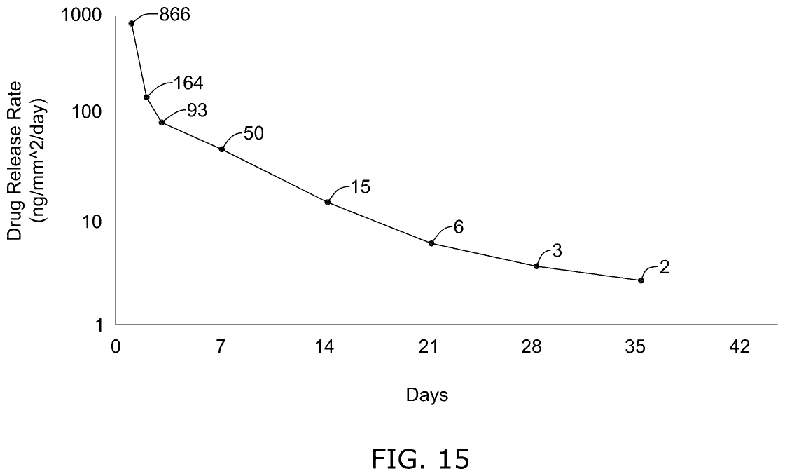

An occlusive implant (e.g., including an expandable framework and an occlusive member) was spray coated with a solution of rivaroxaban and PVDF-HFP (10:90 rivaroxaban:PVDF ratio, solvents: 30:70 acetone:DMF) to achieve an average dose density of about 1.8 μg/mm² on the outer face of the occlusive implant. FIG. 15 shows that there was a significant burst of release drug over the first three days, followed by a decay over the next month. The drug release rate never dropped below 10 ng/mm²/day within the first 14 days.

The spray coated occlusive implants were implanted for 90 days in the left atrial appendages of three different canines. The canines were given no antiplatelet and no anticoagulant medication post-implant. TEE images from each dog 14 days post-implant displayed no evidence of thrombus on any of the occlusive implants, indicating that the rivaroxaban is significantly impacting the local formation of provisional thrombus on the occlusive implants. Rivaroxaban blood content analysis showed that the systemic concentration of rivaroxaban in all the canines was undetectable beyond 24 hours, indicating that the thrombus inhibition was strictly due to local action of the drug, not due to systemic effects of the drug in the bloodstream. CT images were obtained at 45 days post-implant, showing minimal occlusion in Canine 1 (high contrast inflow) and good occlusion in Canine 2 and Canine 3 (minimal contrast inflow through the device mesh). Examination of explanted occlusive implants after 90 days indicated Canine 1 exhibited nearly 100% tissue coverage, Canine 2 exhibited about 90% tissue coverage and Canine 3 exhibited about 70% tissue coverage. The results indicate that a sustained drug release of ≥10 ng/mm²/day for at least 14 days, along with a fast decay in drug release beyond 14 days, yields almost complete tissue coverage at 90 days.

### Example 5—Very Low Dose, Very Fast Release Occlusive Implant

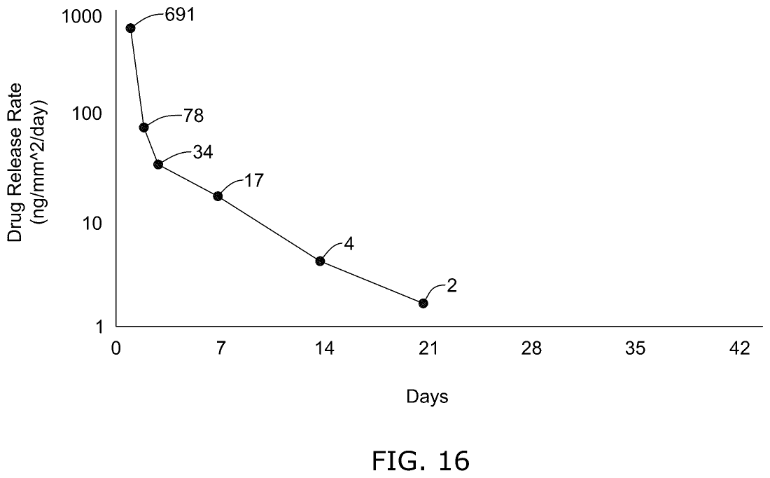

An occlusive implant (e.g., including an expandable framework and an occlusive member) was spray coated with a solution of rivaroxaban and PVDF-HFP (10:90 rivaroxaban:PVDF ratio, solvents: 30:70 acetone:DMF) to achieve an average dose density of about 1.0 μg/mm² on the outer face of the occlusive implant. FIG. 16 shows that there was a significant burst of release drug over the first two days, followed by a decay over the next week. The drug release rate never dropped below 4 ng/mm²/day within the first 14 days.

The spray coated occlusive implants were implanted for 90 days in the left atrial appendages of three different canines. The canines were given no antiplatelet and no anticoagulant medication post-implant. TEE images from each dog 14 days post-implant displayed no evidence of thrombus on any of the occlusive implants, indicating that the rivaroxaban is significantly impacting the local formation of provisional thrombus on the occlusive implants. Rivaroxaban blood content analysis showed that the systemic concentration of rivaroxaban in all the canines was undetectable beyond 24 hours, indicating that the thrombus inhibition was strictly due to local action of the drug, not due to systemic effects of the drug in the bloodstream. CT images were obtained at 45 days post-implant, showing complete occlusion in all three canines at 45 days. Examination of explanted occlusive implants after 90 days indicated Canine 1, Canine 2, and Canine 3 all exhibited nearly 100% tissue coverage. The results indicate that a sustained drug release ≥4 ng/mm²/day for at least 14 days, along with a fast decay in drug release beyond 14 days, yields complete tissue coverage at 90 days.

### Example 6—Low Dose, Low Release Occlusive Implant

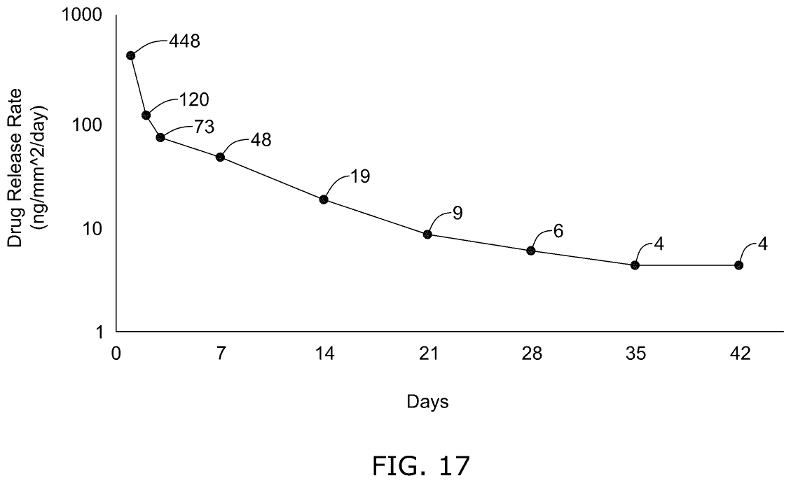

An occlusive implant (e.g., including an expandable framework and an occlusive member) was spray coated with a solution of rivaroxaban and PVDF-HFP (3.75:96.25 rivaroxaban:PVDF ratio, solvents: 16:84 acetone:DMF) to achieve an average dose density of about 1.8 μg/mm² on the outer face of the occlusive implant. FIG. 17 shows that there was a reduced burst of drug release on day 1 compared with formulations with higher drug:polymer ratios, followed by a slower decay over the following weeks. The drug release rate never dropped below 4 ng/mm²/day within the first 42 days.

The spray coated occlusive implants were implanted for 90 days in the left atrial appendages of two different canines. The canines were given no antiplatelet and no anticoagulant medication post-implant. TEE images from each dog 14 days post-implant displayed no evidence of thrombus on any of the occlusive implants, indicating that the rivaroxaban is significantly impacting the local formation of provisional thrombus on the occlusive implants. Rivaroxaban blood content analysis showed that the systemic concentration of rivaroxaban in all the canines was undetectable beyond 24 hours, indicating that the thrombus inhibition was strictly due to local action of the drug, not due to systemic effects of the drug in the bloodstream. CT images were obtained at 45 days post-implant, showing complete occlusion in Canine 1 at 45 days and nearly complete occlusion at 45 days in Canine 2. Examination of explanted occlusive implants after 90 days indicated Canine 1 and Canine 2 exhibited nearly 100% tissue coverage. The results indicate that a sustained drug release ≥4 ng/mm²/day for at least 42 days, along with a slow decay in drug release beyond 42 days, yields complete tissue coverage at 90 days.

### Summary of Examples 1 to 6

In some instances, too high of a drug release rate results in little to no healing. In some instances, too low of a drug release rate results in too much thrombus formation. In some instances, there may be a trade-off between wanting to encourage tissue growth while limiting excessive thrombus formation. FIG. 12 (Example 1) shows a release rate of 618 ng/mm²/day measured 7 days post-implantation, which is too high for tissue growth. FIG. 13 (Example 2) shows a release rate of 178 ng/mm²/day measured 7 days post-implantation, which yields partial coverage and provides an indication of a desired upper drug release rate. FIG. 14 (Example 3) shows a release rate of 59 ng/mm²/day measured 7 days post-implantation. FIG. 15 (Example 4) shows a release rate of 50 ng/mm²/day measured 7 days post-implantation. FIG. 16 (Example 5) shows a release rate of 17 ng/mm²/day measured 7 days post-implantation, and yields 100 percent tissue coverage. FIG. 17 (Example 6) shows a release rate of 48 ng/mm²/day measured 7 days post-implantation. Accordingly, the experimental data supports a desired drug release rate that is in a range of 5 ng/mm²/day to 250 ng/mm²/day measured at 7 days post-implantation. In some instances, a desired drug release rate may be in a range of 50 ng/mm²/day to 200 ng/mm²/day measured at 7 days post-implantation

### Example 7—In-Vitro Evaluation of Other Factor Ca Inhibitors (DOAC)

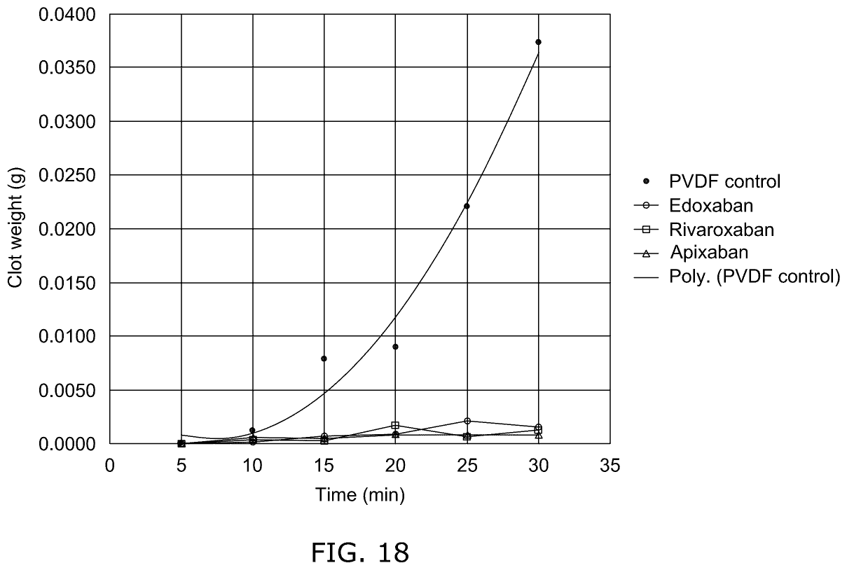

A solution of PVDF-HFP and DOAC (direct oral anticoagulant) was prepared in 80/20 acetone/DMSO (wt/wt). The PVDF-HFP drug ratio was 90/10 (wt/wt) and the solution solids was 0.7%. The DOAC drugs evaluated were Apixaban (Eliquis), Rivaroxaban (Xarelto) and Edxoaban (Savaysa). 15 μmm diameter WM PET fabric disks were dip coated into the polymer/drug solution at a dip speed of 5 μmm/sec. The coated disks were dried for 30 μmin at 125° C. in a convection oven. The drug coated disks and PVDF-HFP only control disks were placed in cups containing heparinized bovine blood adjusted to an ACT of about 190 using protamine. The cups were placed on an orbital shaker incubator at 37° C. and disks were removed at various time points and imaged. The disks were then dried and weighed to determine clot wt. Clot weights are shown in FIG. 18. All three drugs showed significantly less thrombus compared to the PVDF-HFP coated control, showing that the drugs are highly effective at preventing acute thrombus from forming on the occlusive implant fabric.

### Example 8—Other Polymer Carriers for Rivaroxaban

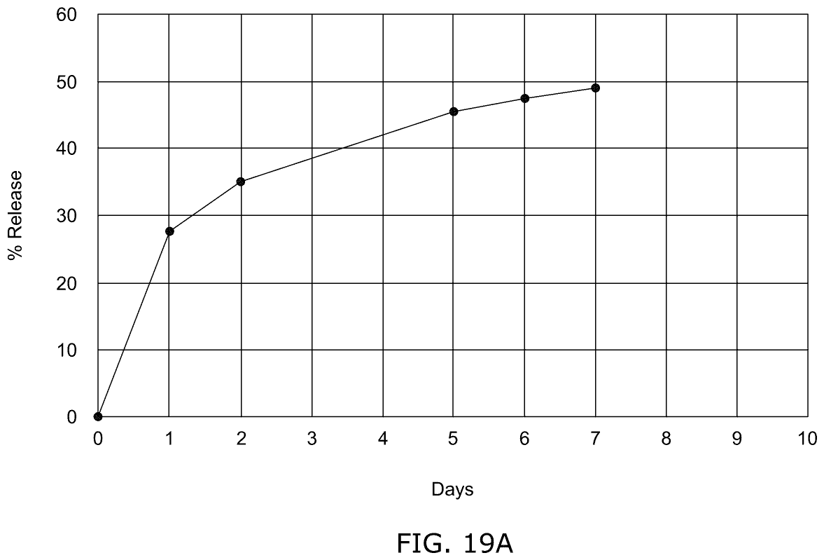

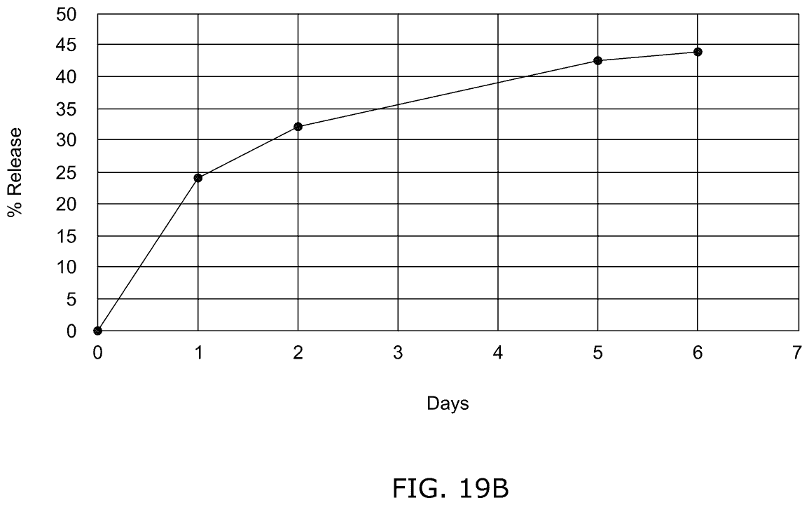

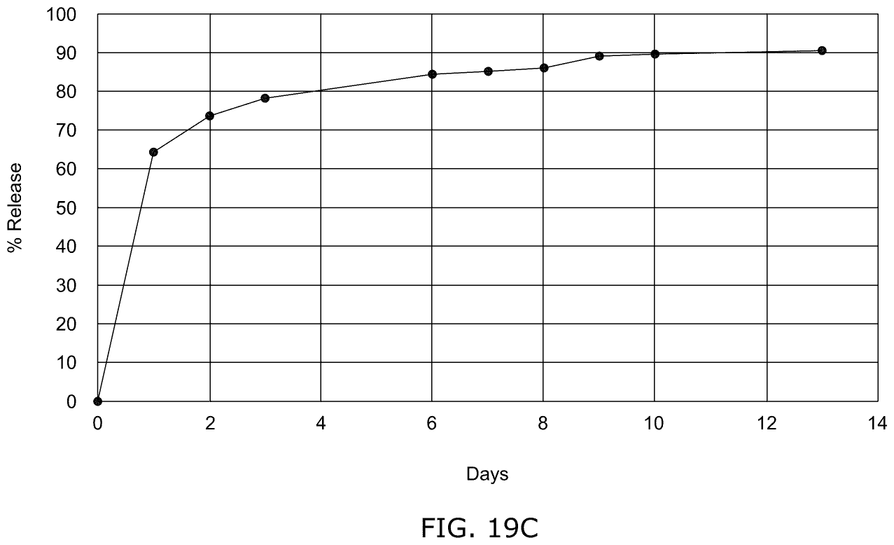

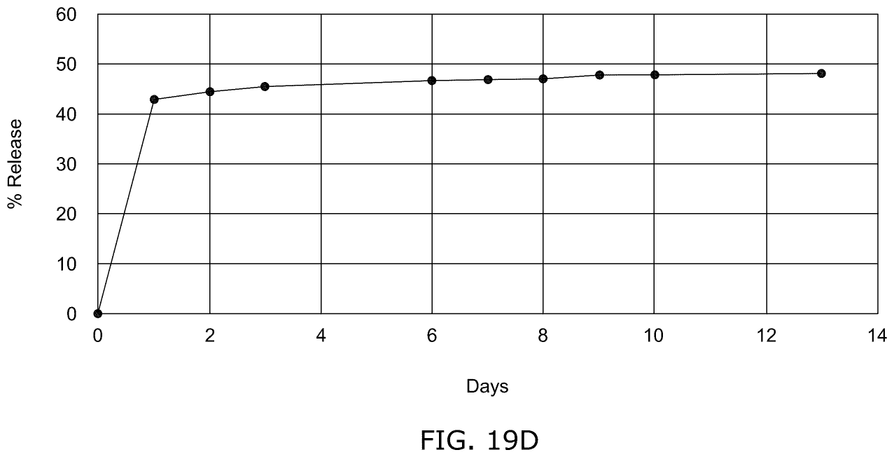

Occlusive implants were coated with other polymer/rivaroxaban formulations. Styrene-isobutylenestyrene block terpolymer (SIBS) and polybutylmethacrylate (PBMA) were formulated with Rivaroxaban and coated on occlusive implants. In-vitro drug release in PBS/tween20 μmedia at 37° C. was measured. Table 1 below shows the formulations evaluated. Coatings were applied via dip coating to the top face of 31 μmm occlusive implants. FIG. 19A shows the drug release curve for Sample 1, FIG. 19B shows the drug release curve for Sample 2, FIG. 19c shows the drug release curve for Sample 3 and FIG. 19d shows the drug release curve for Sample 4. The drug release curves show that both PBMA and SIBS are effective at modulating the release of drugs from the coated occlusive implant.

TABLE 1
Alternative formulations

| Sample | Base Coat | Top Coat |
|---|---|---|
| 1 | 90/10 PBMA/Rivaroxaban (8 mg ct wt) | PVDF-HFP (45 mg) |
| 2 | 90/10 SIBS/Rivaroxaban (9 mg) | PVDF-HFP (6 mg) |
| 3 | 100% Rivaroxaban (0.5 mg) | SIBS (5 mg) |
| 4 | 100% Rivaroxaban (0.5 mg) | None |

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. An occlusive medical device, comprising:
an expandable frame configured to shift between a first configuration and an expanded configuration;
a fabric disposed along at least a portion of the expandable frame; and
a drug eluting coating disposed on the fabric;
wherein the drug eluting coating includes a pharmaceutically active component dispersed within a polymeric carrier.

2. The occlusive medical device of claim 1, wherein the drug eluting coating comprises 1 wt. % to 50 wt. % pharmaceutically active component and 99 wt. % to 50 wt. % polymeric carrier.

3. The occlusive medical device of claim 1, wherein the drug eluting coating comprises a drug density ranging from 0.1 μg/mm² to 20 μg/mm².

4. The occlusive medical device of claim 3, wherein the drug eluting coating comprises more than 10 wt. % pharmaceutically active component and less than 90 wt. % polymeric carrier.

5. The occlusive medical device of claim 4, wherein the drug eluting coating comprises a drug density ranging from 5 μg/mm² to 20 μg/mm².

6. The occlusive medical device of claim 1, wherein the drug eluting coating comprises less than 10 wt. % pharmaceutically active component and more than 90 wt. % polymeric carrier.

7. The occlusive medical device of claim 6, wherein the drug eluting coating comprises a drug density ranging from 0.1 μg/mm² to 2 μg/mm².

8. The occlusive medical device of claim 1, wherein the pharmaceutically active component comprises an anticoagulant drug.

9. The occlusive medical device of claim 1, wherein the pharmaceutically active component comprises rivaroxaban.

10. The occlusive medical device of claim 1, wherein the polymeric carrier comprises a polyvinylidene fluoride copolymer.

11. The occlusive medical device of claim 1, wherein the polymeric carrier comprises poly(vinylidene fluoride-co-hexafluoropropylene).

12. The occlusive medical device of claim 1, further comprising a polymeric top coat disposed over the drug eluting composition.

13. The occlusive medical device of claim 12, wherein the polymeric top coat comprises a polyvinylidene fluoride copolymer.

14. The occlusive medical device of claim 1, wherein the drug eluting coating is adapted to provide a release rate in a range of 5 ng/mm²/day to 250 ng/mm²/day measured at 7 days post-implantation.

15. An occlusive medical device, comprising:
an expandable frame configured to shift between a first configuration and an expanded configuration;
a fabric disposed along at least a portion of the expandable frame; and
a drug eluting coating disposed on the fabric, the drug eluting coating comprising rivaroxaban dispersed within a polyvinylidene fluoride copolymer; and
a polymeric top coat disposed over the drug eluting coating.

16. The occlusive medical device of claim 15, wherein the drug coating comprises rivaroxaban dispersed within poly(vinylidene fluoride-co-hexafluoropropylene).

17. The occlusive medical device of claim 15, wherein the top coat comprises a polyvinylidene fluoride copolymer.

18. The occlusive medical device of claim 17, wherein the top coat comprises poly(vinylidene fluoride-co-hexafluoropropylene).

19. The occlusive medical device of claim 18, wherein the top coat is substantially free of a pharmaceutically active component.

20. An occlusive medical device, comprising:
an expandable frame configured to shift between a first configuration and an expanded configuration;
a fabric disposed along at least a portion of the expandable frame; and
a drug eluting coating comprising rivaroxaban dispersed within poly(vinylidene fluoride-co-hexafluoropropylene) disposed on the fabric; and
a top coat comprising poly(vinylidene fluoride-co-hexafluoropropylene) disposed over the drug eluting coating.
